(19) United States
(12) Patent Application Publication
(10) Pub. No.: US 2012/0193444 A1
(43) Pub. Date: Aug. 2, 2012

(54) FRAGRANCE-DISPENSING SYSTEM HAVING AN ACTIVATION AND/OR CONSUMPTION INDICATOR

(75) Inventors: Frank Meier, Dusseldorf (DE); Matthias Sunder, Dusseldorf (DE); Matthew Freeborn, Scottsdale, AZ (US)

(73) Assignee: Henkel AG & Co. KGaA, Dusseldorf (DE)

(21) Appl. No.: 13/440,010

(22) Filed: Apr. 5, 2012

Related U.S. Application Data
(63) Continuation of application No. PCT/EP2010/056136, filed on May 6, 2010.

(30) Foreign Application Priority Data
Oct. 8, 2009 (DE) 10 2009 045 481.0

(51) Int. Cl. A61L 9/04

(52) U.S. Cl. 239/44

(57) ABSTRACT

A fragrance-dispensing system comprises a wick and diffuser, wherein the diffuser surface in contact with surrounding air is larger than the wick surface in contact with the air, a container for receiving the wick, and a fragrance preparation in the container, wherein the fragrance is transported by the wick to the diffuser by capillary action. Within the capillary flow path of the fragrant substance preparation through the wick and/or diffuser, at least one repository is provided, through which the fragrant substance preparation can flow and which has at least one dye that can be dissolved by the fragrant substance preparation.

### CROSS-REFERENCE TO RELATED APPLICATIONS

This application is a continuation of PCT Application Serial No. PCT/EP2010/056136, filed on May 6, 2010, which claims priority under 35 U.S.C. §119 to 10 2009 045 481.0 (DE) filed on Oct. 8, 2009. The disclosures PCT/EP2010/056136 and DE 10 2009 045 481.0 are hereby incorporated by reference in their entirety.

### FIELD OF THE INVENTION

The present invention generally relates to a diffusion-driven fragrance-dispensing system having an activation and/or consumption indicator.

### BACKGROUND OF THE INVENTION

Diffusion-driven fragrance-dispensing systems are well known in the prior art. Systems based on a wick and a container filled with a liquid, fragrance-containing preparation are particularly well known, in which the wick is inserted into the container, and the preparation, as a result of the capillary forces of the wick, is carried against gravity to the surface of the wick, from where the thus-transported fragrance is discharged into the surroundings, normally by evaporation.

A disadvantage of such fragrance-dispensing systems is that the consumer cannot recognize whether the system is functioning correctly after its initial activation—that is, it is dispensing as intended into the surroundings—and/or when the system is used up.

Accordingly, the object of the invention is to provide a fragrance-dispensing system that displays to the consumer in an easy way that the fragrance-dispensing system has been correctly activated and/or that the fragrance-dispensing system is used up. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

This object is achieved by a fragrance-dispensing system comprising: (a) at least one diffuser having a capillary pathway therein and a surface in contact with the surrounding air; (b) a wick having a capillary pathway therein and a surface area at least partly exposed to said surrounding air, said wick in contact with said diffuser; (c) at least one repository located in said wick and/or said diffuser; (d) a container for receiving said wick; (e) a fragrance preparation in said container and in contact with said wick; and (f) at least one colorant deposited in said repository, said colorant dissolvable in said fragrance preparation, and wherein (i) said fragrance preparation is transported by means of capillary action against the force of gravity along said pathway from said container through said to said diffuser, said fragrance preparation dissolving said colorant during travel through said pathway; and (ii) said surface of said diffuser in contact with said surrounding air is greater than said surface of said wick exposed to said surrounding air.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The fragrance-dispensing system according to the invention includes a wick and at least one diffuser, wherein the surface area of the diffuser that is in contact with surrounding air is greater than the surface area of the wick that is in contact with the surrounding air. In this way the diffuser causes an increase in the surface area, over which fragrance is dispensed to the surroundings.

The fragrance-dispensing system according to the invention moreover includes a container for receiving the wick, as well as a fragrance preparation that is stored in the container, wherein the fragrance preparation is taken up by means of capillary action against the force of gravity into the wick accommodated in the container and transported through the wick to the diffuser. The diffuser is preferably located on the end of the especially stalk-shaped or cylindrical wick facing away from the container.

Within the capillary flow path of the fragrance preparation through the wick and/or diffuser, at least one repository is provided, through which the fragrance preparation can flow and which has at least one colorant that can be dissolved by the fragrance preparation.

In the context of this application, repository means a defined volumetric space inside the fragrance-dispensing system in which a colorant is stored.

The colorant present in the repository is dissolved by the fragrance preparation as it flows through the repository, in particular due to capillary action, and stains with the transported colorant of the fragrance preparation as long as the diffusion path continues through the wick and/or the diffuser.

The wick and/or the diffuser is/are preferably made of a synthetic material and/or a fibrous material of vegetal origin, especially a cellulose-based material. It is of course possible that the wick and the diffuser are produced from the same or different materials.

More advantageously, the diffuser has an essentially circular base in top view, such that the distance between the wick that is connected especially in the center of the circular base with the diffuser, and the external edge of the diffuser is essentially constant. This results in essentially similar diffusion path lengths and times across the base.

In order to produce a higher release of fragrance, then it is advantageous that the ratio of the surface of the diffuser in contact with the surrounding air and the surface of the wick in contact with the surrounding air is between 1000:1 and 1.25:1, preferably between 125:1 and 2.5:1.

The diffuser is preferably formed from a plurality of essentially similar lamellae, wherein one end of the lamella is fixed in or on the wick. However, according to an advantageous further development of the invention, it is also conceivable that the diffuser is formed from at least two groups of various lamellae that differ from one another.

The number of lamellae is in particular at least 6, preferably at least 50, particularly preferably at least 100.

In another particularly advantageous development of the invention, the lamellae each have a surface, consisting of upper and lower sides of a lamella, from 0.5-100 cm², preferably from 1-75 cm², and particularly preferably from 2-50 cm².

It is also possible for the lamellae to be arranged helically (acyclically) or whorled (cyclically) on the wick axis.

The angle between two neighboring lamellae is preferably between 3°-140°, preferably between 4°-137.5°.

In another development of the invention, the diffuser formed from the lamellae can have at least one, preferably two, particularly preferably three planes of symmetry.

The person skilled in the art can use suitable colorants from the prior art for the repository.

Furthermore, it is advantageous for the repository in the not yet accommodated state in the container to be arranged in the wick and/or diffuser such that it cannot be seen from outside.

In principle, it is also conceivable to provide color repositories, each with colorants that differ from one another, in the wick and diffuser.

The colorant can be introduced into the wick and/or diffuser by methods from the prior art, known to the person skilled in the art, for example by infusion (injection), such that a repository of colorant is formed there.

The invention will be explained below in more detail by means of an example.

In a first embodiment, the fragrance-dispensing system consists of a container for a perfume oil-containing fragrance preparation, a wick, a diffuser located on the wick and a perfume oil-containing fragrance preparation for use with the fragrance-dispensing system.

A glass vessel having a ca. 1.5 cm wide opening was used as the container. In principle however, porcelain or ceramic vessels are also suitable.

Any preparations that were developed for wick systems for perfuming rooms can be used as the perfume oil-containing fragrance preparation, e.g. from perfume companies, such as Bell Flavors and Fragrances, Firmenich, Givaudan, International Fragrance & Flavors, Symrise or Takasago. The perfume oil content in the solvent is normally between 2-10% in these types of fragrance preparations.

The usual colorants used in washing or cleaning agents, for example can be used as the colorants.

A water/ethanol mixture is particularly suitable as the solvent for the fragrance-containing preparation. However, isopropanol, dipropylene glycol, diethyl phthalate, isopropyl myristate, benzyl benzoate, 2-(2-ethoxy-ethoxy)-1-ethanol or ethyl citrate, alone or as a mixture, can also be used.

A natural vegetal material is particularly suitable as the wick and/or diffuser material. The vegetal material used is Aeschynomene aspera from the Fabaceae family.

The length of the wick is preferably ca. 8 cm for a diameter of ca. 1 cm. The diameter of the wick is selected such that it can be easily inserted into the opening of the container used to store the fragrance-containing preparation, and the diffuser protrudes several centimeters out of the opening. The container and the wick as well as the diffuser are designed and matched to each other, such that the container with the inserted wick and diffuser is stable. The diameter of the diffuser is ca. 5 cm.

Exemplary colorants can be selected from the following list, which designates the colorants in the C.I. (Color Index) with their generic names: Solvent Red 135, Acid red 18, Food red 7, Azo complex, Pigment Red 112, Pigment violet 23, Basic violet, Acid red 52, C.I. Food Yellow 3, Food red 9, Pigment red 112, FD&C yellow 5, Aluminum lake, Pigment yellow 1, Sunset yellow, Acid Yellow 5, Basic Yellow 28,Yellow 97, DC yellow 8, Acid green 25, Green 7, Patent Blue AE E133, Direct blue 86, Acid Blue 182, Acid blue 9, Acid blue 3, Basic blue 3, Acid blue 225, Blue 15: 1, Blue 15:3, Acid blue 80, Acid blue 7, Acid blue 9, FD & C blue No. 1, Food Blue 2, VAT Red 1, Acid red 27, Solvent yellow 16, Pigment Yellow 1, Acid Yellow 9, Pigment green 7, Pigment blue 15: 1, Pigment blue 15, Acid blue 9, Acid red 52, Basic Violet 10, Basic Violet 10, Basic violet 10, Basic violet 10, Reactive Red 24: 1, Acid yellow 232, Acid red 274, Acid brown 413, Acid red 414, Acid red 52, Acid red 18, Food red 7, Acid red 52, Basic violet 10, FD&C No 40, Food Red 17, Acid red 1, Food red 10, Solvent yellow 179, Disperse yellow 201, Acid yellow 36, Pigment yellow 147, Acid yellow 17, Acid orange 7, Yellow 81, Reactive yellow 161, Food yellow 3, Acid yellow 218, Solvent yellow 174, Acid yellow 3, Reactive yellow 25, Reactive yellow 25, Food yellow 13, Solvent yellow 93, Solvent Orange, Pigment green 7, Solvent Green 3, Solvent Green 7, Pigment green 7, Pigment Green 7, Pigment Green 7, Acid green 25, Food Yellow 4, Acid Yellow 23, Direct blue 86, FD&C Blue No.1, Solvent blue 35, Acid blue 9, Acid Blue 104, Pigment blue 15: 1, Acid blue 182, Solvent Blue 35, Acid blue 182, Acid blue 9, Blue 35, Reactive blue 197, Pigment blue 29, Pigment Blue 15: 1, Acid blue 9, Food Blue 2, and Acid blue 9.

The colorant is added to the inner plies of the wick, for example by impregnating these plies with the colorant or by injecting the colorant into these plies. The external plies are preferably not provided with the colorant of the inner plies, such that a consumer does not see the colorant comprised in the inner plies of the wick, or cannot come into contact with it when using the wick.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. A fragrance-dispensing system comprising:
a. at least one diffuser having a capillary pathway therein and a surface in contact with the surrounding air;
b. a wick having a capillary pathway therein and a surface area at least partly exposed to said surrounding air, said wick in contact with said diffuser;
c. at least one repository located in said wick and/or said diffuser along said capillary pathway;
d. a container for receiving said wick;
e. a fragrance preparation in said container and in contact with said wick; and
f. at least one colorant deposited in said repository, said colorant dissolvable in said fragrance preparation,
wherein (i) said fragrance preparation is transported by means of capillary action against the force of gravity along said capillary pathway from said container through said wick to said diffuser, said fragrance preparation dissolving said colorant during travel through said pathway; and (ii) said surface of said diffuser in contact with said surrounding air is greater than said surface of said wick exposed to said surrounding air.

2. The system according to claim 1, wherein the at least one repository is arranged in the wick and/or diffuser such that it cannot be seen from outside the system prior to the start of capillary flow of said fragrance preparation.

3. The system according to claim 1, wherein said wick or said diffuser comprise a synthetic material or a fibrous material of vegetal origin.

4. The system according to claim 1, wherein said wick or said diffuser comprise a cellulosic material.

5. The system according to claim 1, wherein said diffuser comprises an essentially circular base when viewed from the top.

6. The system according to claim 1, wherein the ratio of the surface of the diffuser in contact with the surrounding air and the surface of the wick in contact with the surrounding air is between 1000:1 and 1.25:1.

7. The system according to claim 6, wherein said ratio is between 125:1 and 2.5:1.

8. The system according to claim 1, wherein the diffuser comprises a plurality of essentially similar lamellae, wherein one end of the lamellae is fixed in or on the wick.

9. The system according to claim 8, wherein the diffuser is formed from at least two groups of lamellae that differ from each other.

10. The system according to claim 9, wherein the number of the lamellae is at least 6.

11. The system according to claim 10, wherein the number of said lamellae is at least 100.

12. The system according to claim 11, wherein the lamellae each have a surface consisting of upper and lower sides of a lamella, said surface having area from 0.5-100 cm².

13. The system according to claim 12, wherein said surface of a lamella is from 2-50 cm².

14. The system according to claim 8, wherein the lamellae are arranged helically or whorled on the wick axis.

15. The system according to claim 8, wherein the angle between two neighboring lamellae is between 3°-140°.

16. The system according to claim 15, wherein said angle is between 4°-137.5°.

17. The system according to claim 8, wherein the diffuser has at least one plane of symmetry.

18. The system according to claim 17, wherein said diffuser has at least two planes of symmetry.

19. The system according to claim 18, wherein said diffuser has at least three planes of symmetry.

20. The system according to claim 1, wherein at least one repository is located in said wick and at least one repository is located in said diffuser, and wherein the at least one colorant deposited in the repository located in said wick differs from the at least one colorant deposited in the repository located in said diffuser.
